(19) United States
(12) Patent Application Publication
(10) Pub. No.: US 2023/0407223 A1
(43) Pub. Date: Dec. 21, 2023

(54) HIGH THROUGHPUT MICRO-WELL ARRAY PLATES AND METHODS OF FABRICATION

(71) Applicant: The Board of Regents of The University of Texas System, Austin, TX (US)

(72) Inventors: Robert M. BACHOO, Dallas, TX (US); Digant P. DAVÉ, Dallas, TX (US)

(73) Assignee: The Board of Regents of The University of Texas System, Austin, TX (US)

(21) Appl. No.: 18/249,888

(22) PCT Filed: Oct. 22, 2021
(86) PCT No.: PCT/US2021/056195

§ 371 Date: Apr. 20, 2023

Related U.S. Application Data
(60) Provisional application No. 63/104,105, filed on Oct. 22, 2020.

(51) Int. Cl. C12M 1/12; C12M 1/00

(52) U.S. Cl. CPC C12M 25/04; C12M 23/38

(57) ABSTRACT

A Micro-Well Array Plates (MWAP) assembly for high throughput microfluidic devices for studying cells and method of manufacturing thereof are provided. The MWAP assembly includes a top plate having a plurality of macro-wells arranged in an array within a frame. The MWAP assembly also includes a bottom plate operable to be secured to the bottom surface of the frame, the bottom plate having a plurality of arrays of micro-wells. The MWAP assembly includes a well grid formed when the bottom plate is secured to the top plate via the plurality of macro-wells and the plurality of arrays micro-wells. The well grid with the plurality of macro-wells and the plurality of arrays micro-wells enable visualization of cells via a high throughput.

### CROSS-REFERENCE TO RELATED APPLICATION

This application claims the benefit of U.S. Provisional Patent Application Ser. No. 63/104,105 filed on Oct. 22, 2020, the content of which is incorporated by reference in its entirety.

### BACKGROUND

### 1. Field

The present inventive concept is directed to cell culture plates, with a high density array of micro-wells for single cell applications and multicellular applications, and fabrication methods thereof. The cell culture plates can be used for tumor and cancer drug screening.

### 2. Discussion of Related Art

Drug development is a time consuming and prohibitively expensive process. High failure rates of tumor drugs can be attributed, in part, to poor selectivity of drug molecules during in-vitro screening. Conventional devices for screening tumor drugs suffer from poor in-vitro drug screening sensitivity.

Conventional micro-well devices for testing drugs are limited to single-cell applications and multi-cellular applications. These conventional devices suffer from low throughput, among other deficiencies.

Accordingly, there is a need to develop apparatuses and associated techniques for high throughput drug and cancer screening applications that do not suffer from the aforementioned deficiencies, have improved in-vitro drug screen sensitivity, are adaptable to accommodate a variety of different application requirements, and are efficient, economical, and easy to fabricate and utilize.

### BRIEF SUMMARY

The present inventive concept provides multiple Micro-Well Array Plates (MWAP) assemblies operable to function as high throughput microfluidic devices for studying cells, methods to fabricate the microfluidic devices, and image analysis procedures using the MWAP assemblies.

The aforementioned may be achieved in one aspect of the present inventive concept by forming a MWAP assembly defined by a top plate and a bottom plate. The top plate has a plurality of macro-wells arranged in an array, within a frame defined by the top plate. The array includes a plurality of columns and a plurality of rows. The bottom plate has a plurality of micro-wells arranged in an array. The bottom plate is operable to be secured to a bottom surface of the frame of the top plate. The MWAP assembly includes a well grid formed when the bottom plate is secured to the top plate. The well grid is defined via the plurality of macro-wells and the plurality of micro-wells with each of the plurality of macro-wells isolating a set of the plurality of micro-wells from another set of the plurality of micro-wells. The bottom plate and/or the top plate may be micro-fabricated plates operable for use as cell culture plates. The plurality of micro-wells form a high density array of microfluidic wells of various shapes and sizes operable to confine single cells or multiple cells. The top plate and the bottom plate are joined or bonded together so that the plates are permanently secured together, e.g., via laser welding the plates together, or selectively detachable from each other, e.g., via joining the plates using a friction-fit engagement and/or a reusable adhesive. The plurality of micro-wells are formed relative to each other to provide varying degrees of spatial confinement asymmetry. In some variations, the top plate may be a bottomless frame with partitioned sections, which may have the same size of a single well in a standard ninety-six (96) well plate. In some variations, the bottom plate may be formed of a micro-fabricated optically clear sheet, and contains an array of micro-wells corresponding to a size and shape of one or more of the plurality of macro-wells.

The disclosure provides different MWAP assemblies, e.g., a large array format and a small array format. In some embodiments, the large array format MWAP assembly, with an array of ninety-six (96) sets of micro-wells arranged in an 8×12 pattern, is dimensionally of a same size as an industry standard ninety-six (96) well plate for cell culture. One application of the MWAP assembly in the large array format is for high throughput. In some embodiments, the small array format MWAP assembly, with an array four (4) sets of macro-wells arranged in a 2×2 pattern, has a same size as a standard microscope slide. The small array format MWAP assembly is designed for low throughput basic research applications to study cells. The MWAP assemblies provide assay protocol for various in-vitro diagnostic and screening applications, including use of the MWAP assemblies to induce tumor cell dormancy, and to produce clonal spheroid of a fixed size.

Additional aspects, advantages, and utilities of the present inventive concept will be set forth, in part, in the description which follows and, in part, will be obvious from the description, or may be learned by practice of the present inventive concept.

The foregoing is intended to be illustrative and is not meant in a limiting sense. Many features and subcombinations of the present inventive concept may be made and will be readily evident upon a study of the following specification and accompanying drawings comprising a part thereof. These features and subcombinations may be employed without reference to other features and subcombinations.

### BRIEF DESCRIPTION OF THE DRAWINGS

The description will be more fully understood with reference to the following figures and data graphs, which are presented as various embodiments of the present inventive concept and should not be construed as a complete recitation of the scope of the present inventive concept, wherein:

FIG. 1A is a bottom perspective view of a top plate of a large format Micro-Well Assay Plates (MWAP) assembly showing ninety-six (96) macro-wells extending through a top, plate prior to assembly of the large format MWAP assembly, in accordance with embodiments of the present inventive concept;

FIG. 1B is a top perspective view of a bottom plate of the large format MWAP assembly showing ninety-six (96) sets of micro-wells in a bottom plate, prior to assembly of the MWAP assembly, in accordance with embodiments of the present inventive concept

FIG. 1C is a top perspective view of the top plate of FIG. 1A, in accordance with embodiments of the present inventive concept;

FIG. 1D is a top perspective view of the top plate and the bottom plate of FIGS. 1A-1C prior to assembly of the large format MWAP assembly, in accordance with embodiments of the present inventive concept;

FIG. 1E is a top plan view of a portion of a single micro-well set of the bottom plate, in accordance with embodiments of the present inventive concept;

FIG. 1F is an elevated side, cross-sectional view of a single macro-well of the top plate and a single array of micro-wells of the bottom plate, after assembly of the large format MWAP assembly to form a portion of a well grid, in accordance with embodiments of the present inventive concept;

FIG. 2A is a top plan view of a single macro-well of the top plate and a single array of micro-wells having varying sizes of the bottom plate, after assembly of the large format MWAP assembly to form a portion of a well grid, in accordance with embodiments of the present inventive concept;

FIG. 2B is a top plan view showing a magnified portion of the single array of micro-wells of the bottom plate of FIG. 2A, in accordance with embodiments of the present inventive concept;

FIG. 2C is a top plan view showing a magnified portion of the single array of micro-wells of the bottom plate of FIG. 2B, in accordance with embodiments of the present inventive concept;

FIG. 3A is a top perspective view of a single micro-well of the bottom plate formed as a square pyramid with a bottom surface, in accordance with embodiments of the present inventive concept;

FIG. 3B is a top plan view of the single micro-well of the bottom plate of FIG. 3A, in accordance with embodiments of the present inventive concept;

FIG. 3C is an elevated side, cross-sectional view of the single micro-well of the bottom plate of FIG. 3A, in accordance with embodiments of the present inventive concept;

FIG. 4A is a top perspective view of a single micro-well of the bottom plate formed as a triangular pyramid with a bottom common point, in accordance with embodiments of the present inventive concept;

FIG. 4B is a top plan view of the single micro-well of the bottom plate of FIG. 4A, in accordance with embodiments of the present inventive concept;

FIG. 4C is an elevated side, cross-sectional view of the single micro-well of the bottom plate of FIG. 4A, in accordance with embodiments of the present inventive concept;

FIG. 5A is a top perspective view of a single micro-well of the bottom plate formed as a square with straight sidewall surfaces, in accordance with embodiments of the present inventive concept;

FIG. 5B is a top plan view of the single micro-well of the bottom plate of FIG. 5A, in accordance with embodiments of the present inventive concept;

FIG. 5C is an elevated side, cross-sectional view of the single micro-well of the bottom plate of FIG. 5A, in accordance with embodiments of the present inventive concept;

FIG. 6A is a bottom perspective view of a top plate of a small format MWAP assembly showing four (4) macro-wells extending through the top plate, prior to assembly of the small format MWAP assembly, in accordance with embodiments of the present inventive concept;

FIG. 6B is a top perspective view of the top plate of FIG. 6A and a bottom plate showing four (4) sets of micro-wells, prior to assembly of the small format MWAP assembly, in accordance with embodiments of the present inventive concept;

FIG. 7 is a flow chart illustrating steps to form the large format MWAP assembly or the small format MWAP assembly, in accordance with embodiments of the present inventive concept;

FIG. 8A is a top perspective view of the large format MWAP assembly with the top plate and the bottom plate of FIGS. 1A-1D being joined together via a welding process, in accordance with embodiments of the present inventive concept;

FIG. 8B is a top perspective view of the small format MWAP assembly with the top plate and the bottom plate of FIGS. 6A-6B being joined together via a welding process, in accordance with embodiments of the present inventive concept;

FIG. 9A is a top view of the bottom plate including pyramid micro-wells for a single tumor cell Dormancy Assay, in accordance with embodiments of the present inventive concept;

FIG. 9B is a fluorescence image showing single cells in the bottom plate for the single tumor cell Dormancy Assay of FIG. 9A, in accordance with embodiments of the present inventive concept;

FIG. 9C is a top view of the bottom plate including the single tumor cells trapped in the pyramid micro-wells with FIGS. 9A and 9B combined, in accordance with embodiments of the present inventive concept;

FIG. 10A is a top view of the bottom plate including square pyramid micro-wells trapped with single clonal cells for a single cell Clonogenic Assay, in accordance with embodiments of the present inventive concept;

FIG. 10B is a top view of the bottom plate including square pyramid micro-wells trapped with colonies of the clonal cells in the single cell Clonogenic Assay of FIG. 10A, in accordance with embodiments of the present inventive concept;

FIG. 11A is a top view of the bottom plate including pyramid micro-wells trapped with single clonal cells for a single cell Clonogenic Assay, in accordance with embodiments of the present inventive concept;

FIG. 11B is a top view of the bottom plate including pyramid micro-wells trapped with colonies of the clonal cells in the single cell Clonogenic Assay of FIG. 11A, in accordance with embodiments of the present inventive concept;

FIG. 12A is a top view of the bottom plate including square pyramid micro-wells trapped with breast cancer cells for a Spheroid Assay, in accordance with embodiments of the present inventive concept; and

FIG. 12B is a top view of the bottom plate including square pyramid micro-wells trapped with grown breast cancer cells in the Spheroid Assay of FIG. 12A, in accordance with embodiments of the present inventive concept.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The illustrations and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

### II. General Architecture

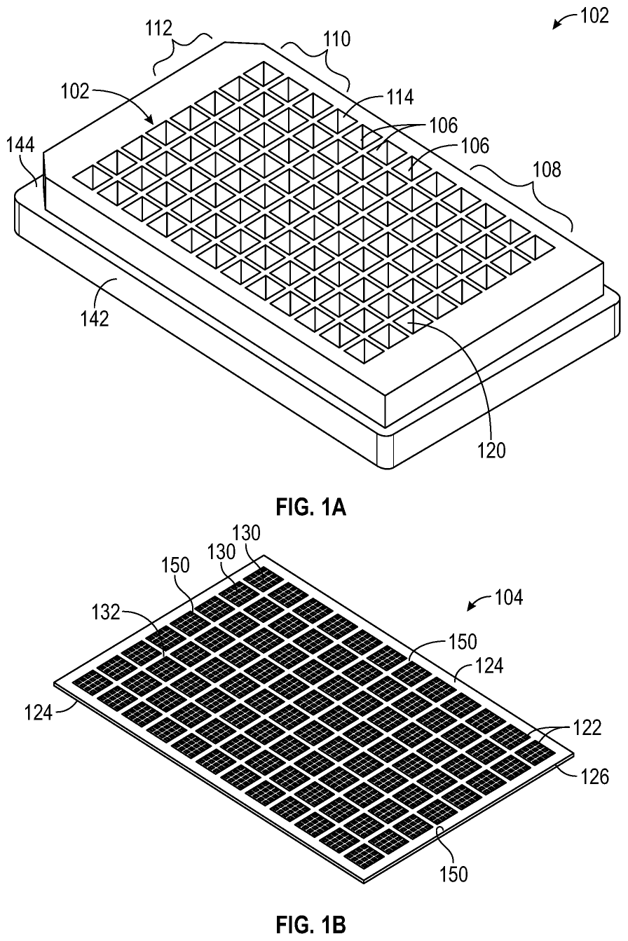

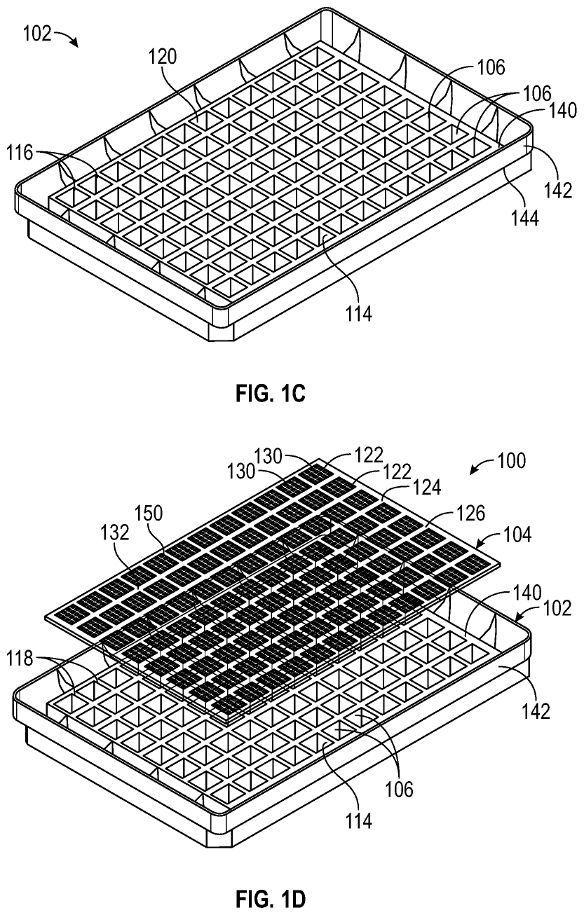

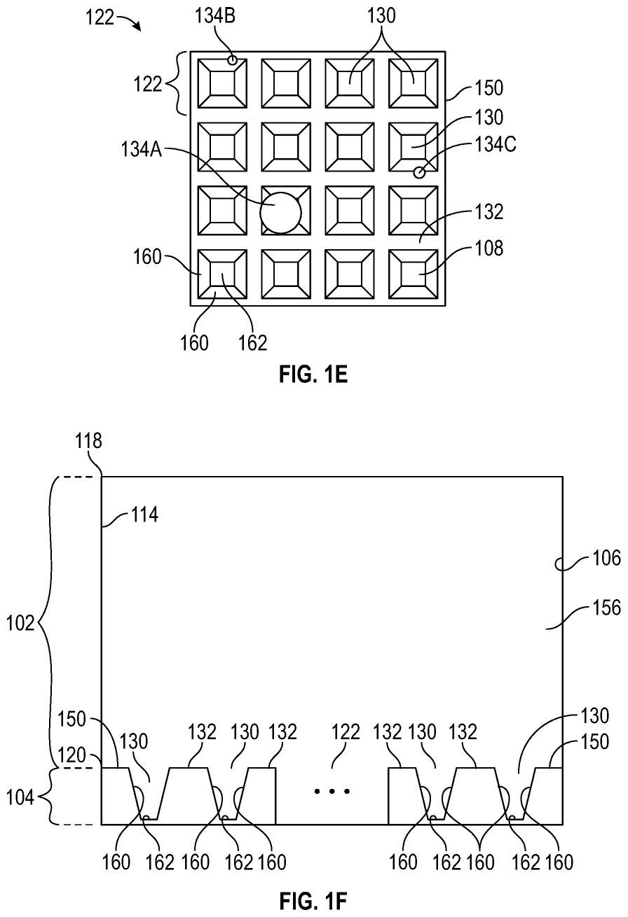

Turning to FIGS. 1A-1F, a large format Micro-Well Assay Plates (MWAP) assembly is illustrated according to an embodiment of the present inventive concept. The MWAP assembly 100 is generally defined by a top plate 102 and a bottom plate 104.

The top plate 102 includes ninety-six (96) macro-wells 106 extending entirely through the top plate 102. The macro-wells 106 are arranged in a macro-well array 108, with eight (8) rows 110 and twelve (12) columns 112. The top plate 102 may also optionally include chamfered corners on one side of the top plate 102. The chamfered corners can serve as alignment markers to facilitate assembly of a cover of a similar shape, e.g., with corresponding chamfered corners, onto the top plate 102 to close one side of the macro-wells 106.

Each of the macro-wells 106 is defined by a perimeter sidewall 114 having an upper peripheral edge 118 and a lower peripheral edge 116. In this manner, the macro-wells are substantially bottomless.

The bottom plate 104 includes ninety-six (96) micro-well sets 122 formed into a substantially planar surface 124 on one side 126 of the bottom plate 104. Each of the micro-well sets 122 includes a micro-well array 128 of micro-wells 130 having various shapes and/or sizes, with the micro-wells 130 spaced from each other by planar surface portions 132. It is foreseen that the micro-well sets 122 may include any number of the micro-wells 130, with different ones of the micro-wells 130 being of different shapes, sizes, and/or patterns, without deviating from the scope of the present inventive concept. Indeed, the number, size, shape, and pattern of the micro-wells 130 shown by the figures are merely for illustrative purposes for understanding the present inventive concept. For example, regarding number of the micro-wells 130, different ones of the micro-well sets 122 may include 3000 to 4000 of the micro-wells 130. As such, the bottom plate 104 may be micro-fabricated to include more than 300,000 of the micro-wells 130. In this manner, the MWAP assembly 100 is advantageously operable to provide high throughput, as further discussed herein. Regarding size and shape of the micro-wells 130, various ones of the micro-wells 130 may by shaped such as, but limited to, one or more cuboids, one or more pyramids, one or more square pyramids, and/or the like. As such, different ones of the micro-wells 130, in different ones of the micro-well sets 122, are advantageously operable to provide varying degrees of three-dimensional (3D) spatial confinement for one or more tumor cells 134A, 134B, 134C placed and stored therein by a user of the MWAP assembly 100. In this manner, the micro-wells 130 advantageously enable the user of the MWAP assembly 100 to study how the one or more tumor cells 134A, 134B, 134C respond to the varying degrees of the 3D spatial confinement. As discussed herein, confining cells serves various functions including isolating and inducing dormancy depending on degree of confinement of the cells. Spatial confinement asymmetry in asymmetrical micro-wells, e.g., asymmetrical pyramid micro-wells is beneficial in stopping cell division. For a cell to divide, symmetrical space is required to separate into equal parts. In a pyramid micro-well, for example, if the cell's surface is touching all sides of the pyramid micro-well, due to dorsal-ventral asymmetry of a pyramid, the cell would be difficult to divide. Since most tumor cells are in a constant state of division, the inability to divide can put the tumor cell into cell cycle arrest, a state of ‘dormancy.’

The bottom plate 104 is secured within a receiver 140 of the top plate 102. The receiver 140 includes a perimeter wall 142 that depends from the top plate 102 and surrounds the bottom plate 104, when the bottom plate 104 is secured to the top plate 102. When the top plate 102 is aligned with the bottom plate 104, each of the macro-wells 106 corresponds to and aligns with one of the micro-well sets 122, with the upper peripheral edge 118 of each of the macro-wells 106 abutting a perimeter abutment surface 150, which surrounds each of the micro-well sets 122 and extends co-planar to the planar surface portions 132. In this manner, the upper peripheral edge 118 of each of the macro-wells 106 and the perimeter abutment surface 150 of each of the micro-well sets 122 function as an interface therebetween.

When the top plate 102 is secured to the bottom plate 104, each of the macro-wells 106 fluidly isolates one of the micro-well sets 122 from another one of the micro-wells sets 122, and a well network or well grid 156 defined via the macro-wells 106 and the micro-wells 130 of the micro-well sets 122 is formed. In this manner, each of the macro-wells 106 is operable to function as a seeding reservoir to guide the one or more tumor cells 134A, 134B, 134C into a selected one of the micro-well sets 122 and into one of the micro-wells 130 of the selected one of the micro-well sets 122, as further discussed herein. Further, each of the macro-wells 106 and an associated one of the micro-well sets 122 are operable to cooperatively isolate the one or more tumor cells 134A, 134B, 134C stored therein. Various environmental conditions such as, but not limited to temperature and/or humidity, may vary across the MWAP assembly 100. As such, the micro-well sets 122 containing the one or more tumor cells 134A, 134B, 134C provide a first isolation zone at a first level closest to the one or more tumor cells 134A, 134B, 134C stored therein, while an associated one of the macro-wells 106 provides a second isolation zone at a second level further away from the one or more tumor cells 134A, 134B, 134C, with the first isolation zone extending between the second isolation zone and the one or more tumor cells 134A, 134B, 134C.

As illustrated, FIGS. 1A and 1C respectively show bottom and top perspective views of the top plate 102 of the MWAP assembly 100 prior to assembly to the bottom plate 104, which is illustrated via FIG. 1B. FIG. 1D is a top perspective view of the top plate 102 aligned with the bottom plate 104 prior to securing the top plate 102 to the bottom plate 104 to form the MWAP assembly 100.

FIG. 1E shows a magnified top plan view of one of the micro-wells 130 of one of the micro-well sets 122 of the bottom plate 104. FIG. 1F shows a magnified cross-sectional views of one of the macro-wells 106 of the top plate 102 secured to and fluidly isolating one of the micro-well sets 122 of the bottom plate 104, after assembly of the MWAP assembly 100. The micro-wells 130 are spaced from each other by the planar surface portions 132. In this example of one of the micro-well sets 122, the micro-wells 130 are illustrated as having a uniform or same pattern of square pyramids. Each of the square pyramids includes a plurality of angled sidewall surfaces 160 and a planar bottom surface 162, which extends perpendicular to the planar surface portions 132. Each of the plurality of angled sidewall surfaces 160 extends between one of the planar surface portions 132 and the planar bottom surface 162 to partially define one of the square pyramids of one of the micro-wells 130. As previously discussed, the micro-well sets 122 may include any number of the micro-wells 130.

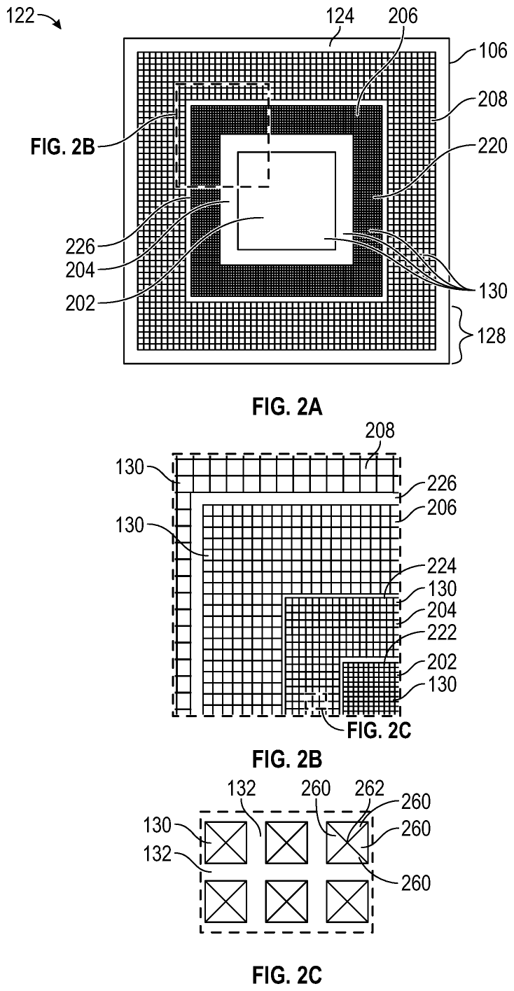

Turning to FIGS. 2A-2C, another one of the macro-wells 106 of the top plate 102 secured to and fluidly isolating another one of the micro-well sets 122 of the bottom plate 104, after assembly of the MWAP assembly 100, is illustrated via different degrees of magnification. As previously discussed, different ones of the micro-wells 130 may be of different shapes, sizes, and/or patterns, without deviating from the scope of the present inventive concept. Regarding different sizes and/or different patterns, FIGS. 2A and 2B illustrate the micro-wells 130 of the one of the micro-well sets 122 as having a plurality of different patterns and a plurality of different shapes. As illustrated, the one of the micro-well sets 122 includes a center area 202 having a square shape, and three (3) concentric areas 204, 206, and 208, which surround the center area 202. The area 208 is an outermost one of the areas 202, 204, 206, and 208, and is directly adjacent to the perimeter abutment surface 150. Divider surfaces 222, 224, 226 extend between different ones of the areas 202, 204, 206, 208 from each other. In this manner, the different patterns of the areas 202, 204, 206, 208 are spaced from each other by the divider surfaces 222, 224, 226.

FIG. 2B is a magnified view of the micro-wells 130 of FIG. 2A. FIG. 2B illustrates the different patterns of the areas 202, 204, 206, 208. As shown, the micro-wells 130, in different ones of the areas 202, 204, 206, 208, are of varying, respectively increasing size. Largest-sized ones of the micro-wells 130 are within the outermost one of the areas 202, 204, 206, and 208, i.e., the area 208, and separated from medium-sized ones of the micro-wells 130 by the divider surface 226. The medium-sized ones of the micro-wells 130 are within the area 206, and surround smaller ones of the micro-wells 130, within the area 204. The medium-sized ones and the smaller ones of the micro-wells 130 are separated from each other by the divider surface 224. The smaller ones of the micro-wells 130 surround smallest ones of the micro-wells 130, within the area 202, and are separated from the smaller ones of the micro-wells 130 by the divider surface 222. It is foreseen that different ones of the micro-wells 130 within one or more of the areas 202, 204, 206, and 208 may have different shapes, in addition to different sizes.

FIG. 2C is a magnified view of six (6) of the micro-wells 130 of FIG. 2B. As shown, the micro-wells 130, in this example, are shaped as pyramids. Each of the pyramids includes a plurality of angled surfaces 260, which extend to a common point 262. Each of the plurality of angled sidewall surfaces 160 extends between one of the planar surface portions 132 and the common point 262 to partially define one of the pyramids of one of the micro-wells 130 in the area 204.

It is foreseen that the micro-wells 130 within a same one of the areas 202, 204, 206, and 208 may have one or more common features, e.g., a same shape, but with different depths and/or different widths, thereby allowing a user to isolate one or more different features and determine how the one or more different features affect the one or more tumor cells 134A, 134B, 134C. For instance, in a particular embodiment, each of areas 202, 204, 206, and 208 include the micro-wells 130 shaped as pyramids, with each of the areas 202, 204, 206, and 208 of a different size, e.g., with the angled sidewall surfaces 160 having respective lengths of 10 μm, 25 μm, 50 μm, and 100 μm.

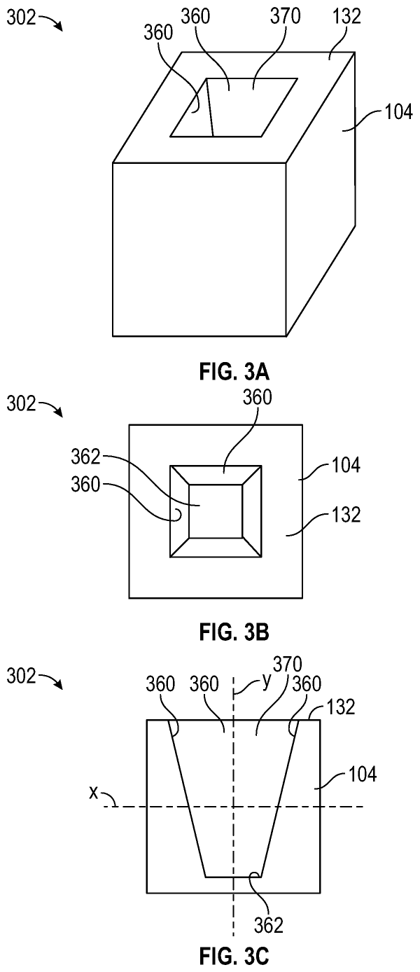

Turning to FIG. 3A, a perspective view of one of the micro-wells 130 of the MWAP assembly 100 is illustrated, with a shape of a square pyramid, i.e., square pyramid micro-well 302. The square pyramid micro-well 302 is defined by a four (4) angled sidewall surfaces 360, which extend obliquely to a planar bottom surface 362, thereby defining an interior 370 of the one of the micro-wells 130, with a width and depth. Each of the plurality of angled sidewall surfaces 360 extends between one of the planar surface portions 132 and the planar bottom surface 362 to partially define the square pyramid micro-well 302 of the one of the micro-wells 130. FIG. 3B is a top plan view of the square pyramid micro-well 302 of FIG. 3A showing each of the four (4) angled sidewall surfaces 360 extending between the planar surface portions 132 and the planar bottom surface 362. FIG. 3C is a cross-sectional view of the square pyramid micro-well 302 of FIG. 3B showing each of the four (4) angled sidewall surfaces 360 and the planar bottom surface 362 extending perpendicular to the planar surface portions 132. As shown in FIG. 3C, the square pyramid micro-well 302 is symmetric with respect to a vertical Y axis and asymmetric with respect to a horizontal X axis. In this manner, when a single cell, e.g., one of the tumor cells 132A, 132B, 132C, is placed into the square pyramid micro-well 302 of the bottom plate 104, along the Y axis, through a corresponding one of the macro-wells 106 of the top plate 102, the single cell experiences the 3D spatial confinement via a plurality of surfaces, e.g., the planar bottom surface 362 and one or more of the four (4) angled sidewall surfaces 360. In this manner, the square pyramid micro-well 302 advantageously enables a user of the MWAP assembly 100 to study how the one or more tumor cells 134A, 134B, 134C respond to the 3D spatial confinement provided by the square pyramid micro-well 302, which may be different than how the one or more tumor cells 134A, 134B, 134C responds in one or more other ones of the micro-wells 130.

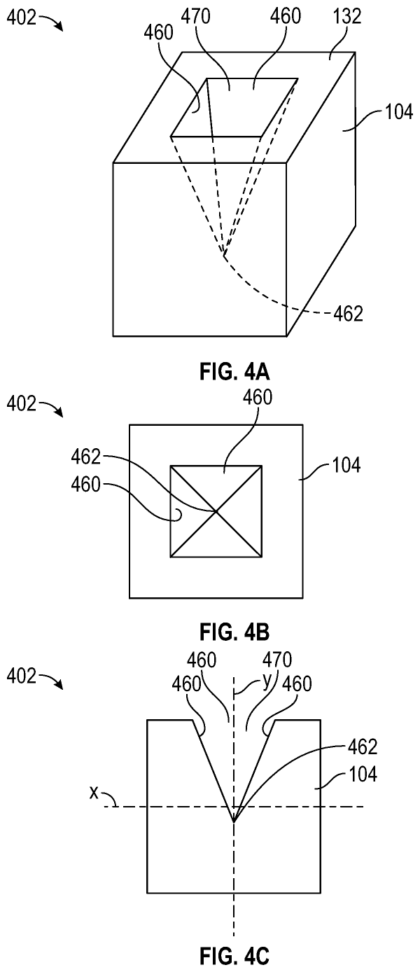

Turning to FIG. 4A, a perspective view of one of the micro-wells 130 of the MWAP assembly 100 is illustrated, with a shape of a pyramid, i.e., pyramid micro-well 402. The pyramid micro-well 402 is defined by a four (4) angled sidewall surfaces 460, which extend to a common bottom point 462, thereby defining an interior 470 of the one of the micro-wells 130, with a width and depth. Each of the plurality of angled sidewall surfaces 460 extends between one of the planar surface portions 132 and the common bottom point 462 to partially define the pyramid micro-well 402 of the one of the micro-wells 130. FIG. 4B is a top plan view of the pyramid micro-well 402 of FIG. 4A showing each of the four (4) angled sidewall surfaces 460 extending between the planar surface portions 132 and the common bottom point 462. FIG. 4C is a cross-sectional view of the pyramid micro-well 402 of FIG. 4B showing each of the four (4) angled sidewall surfaces 460 and the common bottom point 462. As shown in FIG. 4C, the pyramid micro-well 402 is symmetric with respect to a vertical Y axis and asymmetric with respect to a horizontal X axis. Again, when a single cell, e.g., one of the tumor cells 132A, 132B, 132C is placed into the pyramid micro-well 402 of the bottom plate 104, along the Y axis, through a corresponding one of the macro-wells 106 of the top plate 102, the single cell experiences the 3D spatial confinement via a plurality of surfaces, e.g., a plurality of the four (4) angled sidewall surfaces 460. In this manner, the pyramid micro-well 402 advantageously enables the user of the MWAP assembly 100 to study how the one or more tumor cells 134A, 134B, 134C respond to the 3D spatial confinement provided by the pyramid micro-well 402, which may be different than how the one or more tumor cells 134A, 134B, 134C responds in one or more other ones of the micro-wells 130.

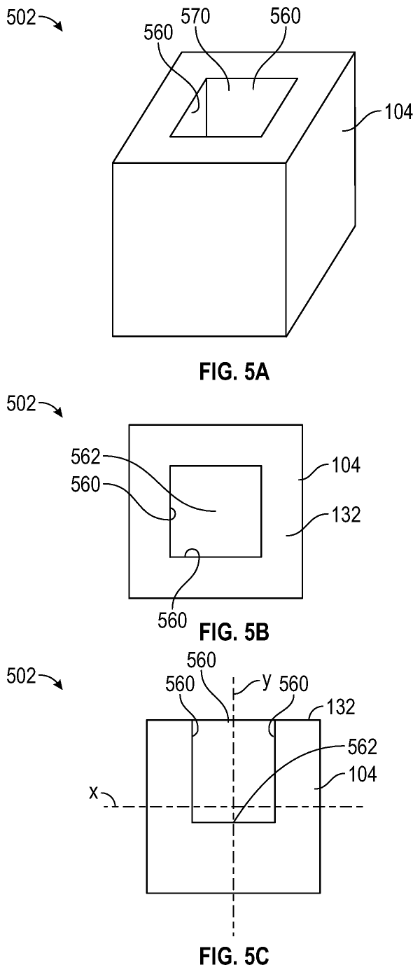

Turning to FIG. 5A, a perspective view of one of the micro-wells 130 of the MWAP assembly 100 is illustrated, with a shape of a square, i.e., square micro-well 502. The square micro-well 502 is defined by a four (4) straight sidewall surfaces 560, which extend perpendicular to a planar bottom surface 562, thereby defining an interior 570 of the one of the micro-wells 130, with a width and depth. Each of the plurality of straight sidewall surfaces 560 extends between one of the planar surface portions 132 and the planar bottom surface 562 to partially define the square micro-well 502 of the one of the micro-wells 130. FIG. 5B is a top plan view of the square micro-well 502 of FIG. 5A showing each of the four (4) straight sidewall surfaces 560 extending between the planar surface portions 132 and the planar bottom surface 562. FIG. 5C is a cross-sectional view of the square micro-well 502 of FIG. 5B showing each of the four (4) straight sidewall surfaces 560 and the planar bottom surface 562 extending perpendicular to the planar surface portions 132. As shown in FIG. 5C, the square micro-well 502 is symmetric with respect to a vertical Y axis and asymmetric with respect to a horizontal X axis. When a single cell, e.g., one of the tumor cells 132A, 132B, 132C is placed into the square micro-well 502 of the bottom plate 104, along the Y axis, through a corresponding one of the macro-wells 106 of the top plate 102, the single cell experiences symmetric confinement, which is not beneficial to stop cell division. In this manner, the square micro-well 502 advantageously enables the user of the MWAP assembly 100 to study and contrast how the one or more tumor cells 134A, 134B, 134C respond to the symmetric confinement provided by the square micro-well 502 relative to how the one or more tumor cells 134A, 134B, 134C responds in one or more other ones of the micro-wells 130. For example, the square micro-well 502 may be used to investigate confinement when a micro-well can hold only one cell laterally, e.g., on an X-Z plane. In this case, the cell has no space to divide laterally, but can potentially divide up along the y-axis. The square micro-well 502 can also be used for a Clonogenic Assay as discussed herein.

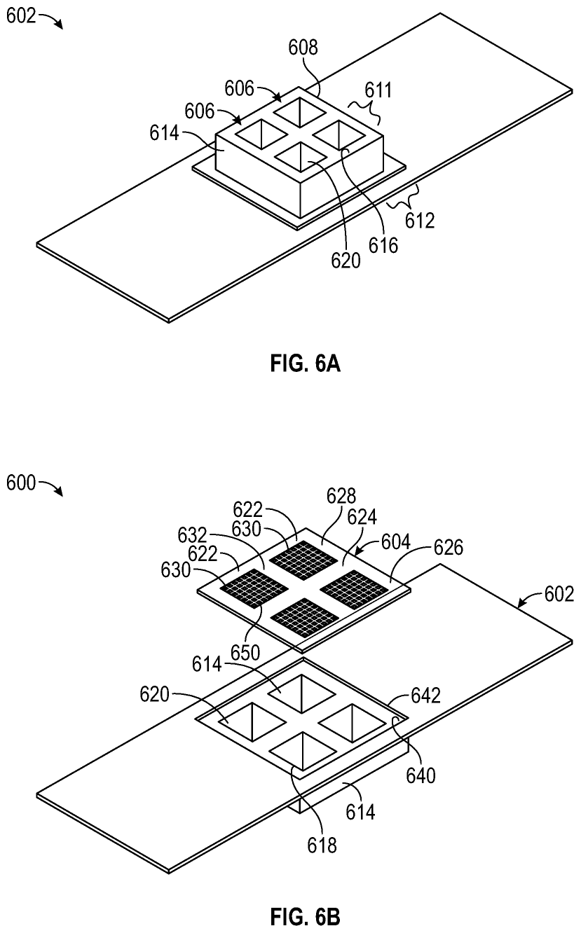

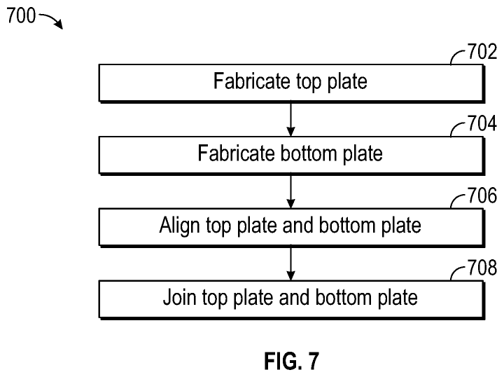

Turning to FIGS. 6A-B, a small format Micro-Well Assay Plates (MWAP) assembly 600 is illustrated according to an embodiment of the present inventive concept. The MWAP assembly 600 is generally defined by a top plate 602 and a bottom plate 604.

The top plate 602 includes four (4) macro-wells 606 extending entirely through the top plate 602. The macro-wells 606 are arranged in a macro-well array 608 with two (2) rows 611 and two (2) columns 612. Each of the macro-wells 606 is defined by a perimeter sidewall 614 with a lower peripheral edge 616 spaced from an upper peripheral edge 618 of the perimeter sidewall 614. The perimeter sidewall 614 defines a reservoir 620 extending entirely through the top plate 602, thereby causing each of the macro-wells 606 to be substantially bottomless.

The bottom plate 604 includes four (4) micro-well sets 622 formed into a substantially planar surface 624 on one side 626 of the bottom plate 604. Each of the micro-well sets 622 includes a micro-well array 628 of micro-wells 630 having various shapes and/or sizes, with the micro-wells 630 spaced from each other by planar surface portions 632. It is foreseen that the micro-well sets 622 may include any number of the micro-wells 630, with different ones of the micro-wells 630 being of different shapes, sizes, and/or patterns, without deviating from the scope of the present inventive concept. Indeed, the number, size, shape, and pattern of the micro-wells 630 shown by the figures are merely for illustrative purposes for understanding the present inventive concept. For example, regarding number of the micro-wells 630, different ones of the micro-well sets 622 may include 3000 to 4000 of the micro-wells 630. As such, the bottom plate 604 may be micro-fabricated to include more than 300,000 of the micro-wells 630. In this manner, the MWAP assembly 600 is advantageously operable to provide high throughput, as discussed herein. Regarding size and shape of the micro-wells 630, various ones of the micro-wells 630 may by shaped such as, but limited to, one or more cuboids, one or more pyramids, one or more square pyramids, and/or the like. As such, different ones of the micro-wells 630, in different ones of the micro-well sets 622, are advantageously operable to provide varying degrees of three-dimensional (3D) spatial confinement for one or more tumor cells, e.g., the one or more tumor cells 134A, 1348, 134C, placed and stored therein by a user of the small format MWAP assembly 600. In this manner, the micro-wells 630 advantageously enable the user of the small format MWAP assembly 600 to study how the one or more tumor cells 134A, 1348, 134C respond to the varying degrees of the 3D spatial confinement. As discussed herein, confining cells serves various functions including isolating and inducing dormancy depending on degree of confinement of the cells. Asymmetric confinement is beneficial to stopping cell division, such as asymmetrical pyramid micro-wells.

The bottom plate 604 is secured within a receiver 640 of the top plate 602. The receiver 640 includes a perimeter wall 642 that depends from the top plate 602 and surrounds the bottom plate 604, when the bottom plate 604 is secured to the top plate 602. When the top plate 602 is aligned with the bottom plate 604, each of the macro-wells 606 corresponds to and aligns with one of the micro-well sets 622, with the upper peripheral edge 618 of each of the macro-wells 106 abutting a perimeter abutment surface 650, which surrounds each of the micro-well sets 622 and extends co-planar to the planar surface portions 132. In this manner, the upper peripheral edge 618 of each of the macro-wells 606 and the perimeter abutment surface 650 of each of the micro-well sets 622 function as an interface therebetween.

When the top plate 602 is secured to the bottom plate 604, each of the macro-wells 606 fluidly isolates one of the micro-well sets 622 from another one of the micro-wells sets 622, and a well network or well grid 656 defined via the macro-wells 606 and the micro-wells 630 of the micro-well sets 622 is formed. In this manner, each of the macro-wells 606 is operable to function as a seeding reservoir to guide the one or more tumor cells 134A, 134B, 134C into a selected one of the micro-well sets 622 and into one of the micro-wells 630 of the selected one of the micro-well sets 622, as further discussed herein. Further, each of the macro-wells 606 and an associated one of the micro-well sets 622 are operable to cooperatively isolate the one or more tumor cells 134A, 134B, 134C stored therein. Various environmental conditions such as, but not limited to temperature and/or humidity, may vary across the small format MWAP assembly 600. As such, the micro-well sets 622 containing the one or more tumor cells 134A, 134B, 134C provide a first isolation zone at a first level closest to the one or more tumor cells 134A, 134B, 134C stored therein, while an associated one of the macro-wells 606 provides a second isolation zone at a second level further away from the one or more tumor cells 134A, 134B, 134C, with the first isolation zone extending between the second isolation zone and the one or more tumor cells 134A, 134B, 134C.

As illustrated, FIG. 6A shows a bottom perspective view of the top plate 602 of the small format MWAP assembly 600 prior to assembly to the bottom plate 604. FIG. 6B is a top perspective view of the top plate 602 aligned with the bottom plate 604 prior to securing the top plate 602 to the bottom plate 604 to form the small format MWAP assembly 600.

### Micro-Well Design

As discussed, the micro-wells 130, 630 may vary in shape and/or size across the bottom plate 104, 604 to enable formation and study of cells of various sizes in a same one of the macro-wells 106, 406. FIGS. 1E, 1F, 2A, 2B, 2C, 3A, 3B, 3C, 4A, 4B, 4C, 5A, 5B, and 5C as previously described herein, with respect to the MWAP assembly 100, are equally applicable to the small format MWAP assembly 600. Indeed, it is foreseen that the features of the MWAP assembly 100 and the small format MWAP assembly 600 including, but not limited to the macro-wells 106, 606 and the micro-wells 130, 630, may be the same without deviating from the scope of the present inventive concept.

In some variations, the bottom plate 104, 604 may have the micro-wells 130, 630 of a square shape, with a same cross-section and a fixed depth, with straight side walls. In some variations, the bottom plate 104, 604 may have the micro-wells 130, 630 of a square shape, with a same cross-section and a fixed depth, with slanted side walls. In some variations, the bottom plate 104, 604 may have the micro-wells 130, 630 of a square shape, with varying cross-sections and varying depths, with slanted side walls. In some variations, the bottom plate 104, 604 may have the micro-wells 130, 630 of a square shape, with varying cross-sections and varying depths, with straight side walls.

In some variations, the bottom plate 104, 604 may have the micro-wells 130, 630 of a triangular shape, with a same cross-section and a fixed depth, with straight side walls. In some variations, the bottom plate 104, 604 may have the micro-wells 130, 630 of a triangular shape, with a same cross-section and a fixed depth, with slanted side walls. In some variations, the bottom plate 104, 604 may have the micro-wells 130, 630 of a triangular shape, with varying cross-sections and varying depths, with straight side walls. In some variations, the bottom plate 104, 604 may have the micro-wells 130, 630 of a triangular shape, with varying cross-sections and varying depths, with slanted side walls.

In some variations, the top plate 102, 602 and/or the bottom plate 104, 604 may be used a polymer or a gel coating of varying stiffness. In some variations, the top plate 102, 602 and/or the bottom plate 104, 604 can be coated with one or more chemistries to modify one or more properties of the top plate 102, 602 and/or the bottom plate 104, 604, e.g., durotactic and/or adherence.

In some variations, a cross-section dimensions of each of the micro-wells 130, 630 may range from 8 μm to 100 μm. For example, a square cross-section of each of the micro-wells 130, 630 may have a side length from 8 μm to 100 μm. An equilateral triangle cross-section of each of the micro-wells 130, 630 may have a side length from 8 μm to 100 μm.

In some variations, small dimensional ones of the micro-wells 130, 630, with a cross-section dimensions of 8 μm to 25 μm, may be used for single cell applications. In some variations, large dimensional ones of the micro-wells 130, 630, with a cross-section dimensions of 26 μm to 100 μm, may be used for multi-cellular applications.

Dimensions of each of the micro-wells 130, 630 may include side length, side width, and/or depth. In some variations, the dimensions of each of the micro-wells 130, 630 may be equal to or greater than 8 μm. In some variations, the dimensions of each of the micro-wells 130, 630 may be equal to or greater than 10 μm. In some variations, the dimensions of each of the micro-wells 130, 630 may be equal to or greater than 15 μm. In some variations, the dimensions of each of the micro-wells 130, 630 may be equal to or greater than 20 μm. In some variations, the dimensions of each of the micro-wells 130, 630 may be equal to or greater than 25 μm. In some variations, the dimensions of each of the micro-wells 130, 630 may be equal to or greater than 30 μm. In some variations, the dimensions of each of the micro-wells 130, 630 may be equal to or greater than 40 μm. In some variations, the dimensions of each of the micro-wells 130, 630 may be equal to or greater than 50 μm. In some variations, the dimensions of each of the micro-wells 130, 630 may be equal to or greater than 60 μm. In some variations, the dimensions of each of the micro-wells 130, 630 may be equal to or greater than 70 μm. In some variations, the dimensions of each of the micro-wells 130, 630 may be equal to or greater than 80 μm. In some variations, the dimensions of each of the micro-wells 130, 630 may be equal to or greater than 90 μm. In some variations, the dimensions of each of the micro-wells 130, 630 may be less than or equal to 100 μm. In some variations, the dimensions of each of the micro-wells 130, 630 may be less than or equal to 90 μm. In some variations, the dimensions of each of the micro-wells 130, 630 may be less than or equal to 80 μm. In some variations, the dimensions of each of the micro-wells 130 may be less than or equal to 70 μm. In some variations, the dimensions of each of the micro-wells 130, 630 may be less than or equal to 60 μm. In some variations, the dimensions of each of the micro-wells 130, 630 may be less than or equal to 50 μm. In some variations, the dimensions of each of the micro-wells 130, 630 may be less than or equal to 40 μm. In some variations, the dimensions of each of the micro-wells 130, 630 may be less than or equal to 30 μm. In some variations, the dimensions of each of the micro-wells 130, 630 may be less than or equal to 25 μm. In some variations, the dimensions of each of the micro-wells 130, 630 may be less than or equal to 20 μm. In some variations, the dimensions of each of the micro-wells 130, 630 may be less than or equal to 15 μm. In some variations, the dimensions of each of the micro-wells 130, 630 may be less than or equal to 10 μm. In some variations, the depth of each of the micro-wells 130, 630 may range from 8 μm to 100 μm. Depth of each of the micro-wells 130, 630 is defined, in part by, sidewalls of each of the micro-wells 130, 630.

### Fabrication of MWAP Assembly

A fabrication process 700 includes forming the top plate 102, 602 using cyclic olefin polymer (COP) of a black color. In this manner, laser welding of the top plate 102, 602 can be facilitated and artifact-free imaging due to reduced reflection and fluorescence can be obtained via the black COP. Using the black COP, the top plate 102, 602 is formed using an injection molding process, at operation 702. Injection molding is a manufacturing process for producing plastic parts by injecting molten material into a mold. For example, the mold may have the pattern of the top plate 102, 602, such as including the macro-wells 106, 606 and the reservoirs 120, 620. COP pellets are fed into a heated barrel and injected into a mold cavity, where the COP cools down to form the top plate 102, 602.

Next, the fabrication process 700 includes forming the bottom plate 104, 604 using an optically clear COP, which provides an optically clear window when viewing the one or more tumor cells 134A, 134B, 134C stored with the micro-wells 130, 630 of the bottom plate 104, 604. In this manner, the bottom plate 104, 604 enables inspection or imaging of cells within the MWAP assembly 100, 600, and analysis of images to visualize cells. Using the optically clear COP, the bottom plate 104, 604 is formed using a hot embossing process, at operation 704. The hot embossing process, which is a process of structuring polymer films or sheets by pressing a stamp into the polymer while the polymer is heated about its glass transition temperature. The polymer is much thicker than the height of the stamp structures. The relief is a perturbation of the total thickness of the polymer. Hot embossing is less prone to defects than nanoimprint lithography and is not limited to nano-structures or micro-structures. First, a silicon master mold is created with the imprint of the desired micro-well sets 122, 622. SU-8 is a commonly-used epoxy-based negative photoresist. Each set of the micro-well sets 122, 622 includes the micro-wells 130, 630, e.g. 3000 to 4000 of the micro-wells 130. The silicon SU-8 master mold is produced using a lithography process of spin coating SU-8 photoresist on a silicon wafer and then by exposure to UV light in a mask aligner. Then, non-cross-linked photoresist is washed away. The silicone SU-8 master mold with features of each micro-well 130, 630 in the micro-well sets 122, 622 is produced. Next, negative stamps of silicone are produced by pouring and peeling silicone on silicon mater. In the final step, the bottom plate 104, 604 is fabricated by transferring or imprinting features on the silicone stamp onto plain sheets of COP by the hot embossing process. In some embodiments, the COP sheets may have a thickness ranging from 100 μm to 800 μm. In some embodiments, the COP sheets may have a thickness ranging from 100 μm to 400 μm. In some embodiments, the COP sheets may be 188 μm thick. In this manner, the bottom plate 104, 604 is micro-fabricated.

Next, the fabrication process 700 includes aligning the top plate 102, 602 and the bottom plate 104, 604, at operation 706. Once aligned, the top plate 102, 602 and the bottom plate 104, 604 are held by fixture and vacuum. For example, the top plate 102, 602 and the bottom plate 104, 604 can be aligned for precision positioning of the micro-well sets 122, 622 with respect to the macro-wells 106, 406, and then held under suction, via a vacuum, on a 3D automated translation stage system during a joining process, at operation 708.

The joining process includes joining or bonding the top plate 102, 602 and the bottom plate 104, 604 together to form the MWAP assembly 100, 600. The top plate 102, 602 and the bottom plate 104, 604 are joined or bonded together and fluidly sealed by a laser welding process at an interface or junction between the top plate 102, 602 and the bottom plate 104, 604 along peripheries of the micro-well sets 122, 622 and the macro-wells 106, 406, as further discussed hereafter.

Laser welding provides a number of advantages over other bonding processes such as gluing. Laser welding is faster than gluing and can also be used for automatic and large scale processes. Further, bonding provided via glue generally has low throughput, and potential complications due to chemical interactions with cells. The laser welding process of the present inventive concept is automatic and faster, and therefore has higher throughput. Further, the laser welding process of the present inventive concept better ensures no leaks will form and does not require chemicals. Other attachment processes such as via a friction-fit engagement and/or gluing provides a number of advantages over welding. For instance, the top plate 102, 602 and the bottom plate 104, 604 can be joined together to form the MWAP assembly 100, 600 so that the top plate 102, 602 is selectively detachable from the bottom plate 104, 604. For example, the joining process may utilize a friction-fit engagement and/or a reusable adhesive to allow the user to selectively attach and detach the top plate 102, 602 from the bottom plate 104, 604. In this manner, the user is advantageously provided with direct access to the cells within the micro-wells 130, 630, thereby allowing further testing and/or inspection.

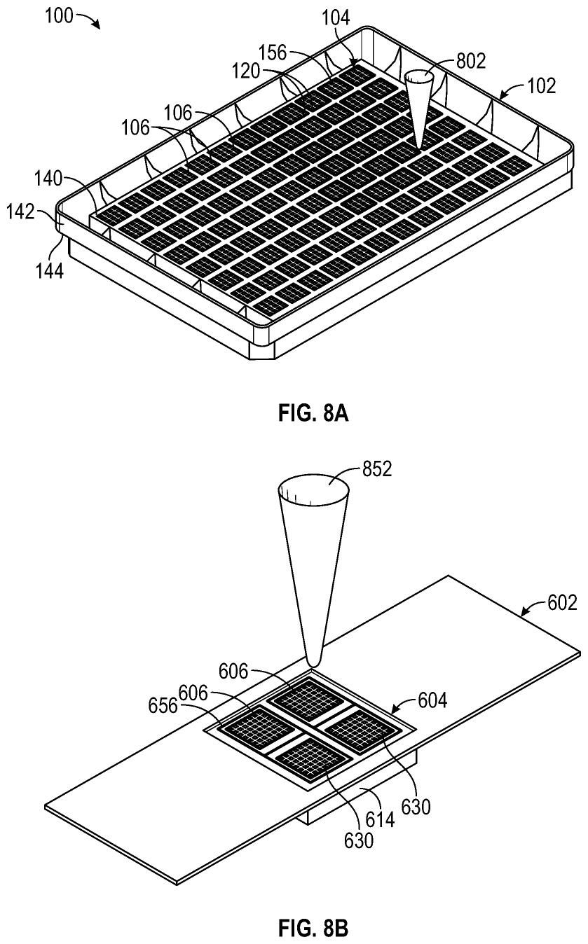

Turning to FIG. 8A, the operation 706 to form the MWAP assembly 100 is illustrated. The top plate 102 and the bottom plate 104 are aligned and held under suction on a 3D automated translation stage system including translation stages during the laser welding process. Next, via the operation 708, a laser spot from a laser source 802 is focused on a first interface between the top plate 102 and the bottom plate 104. Using alignment markers as a reference on the bottom plate 104, the translation stages move in a fixed 2D pattern, with the laser source 802 activated, thereby causing the bottom plate 104 to be welded, joined, and secured to the top plate 102 along each periphery of the macro-wells 106, in a square pattern. In this manner, the top plate 102 and the bottom plate 104 are fluidly sealed to each other, and the MWAP assembly 100, with the well grid 156, is formed.

Turning to FIG. 8B, the operation 506 to form the small format MWAP assembly 600 is illustrated. The top plate 602 and the bottom plate 604 are aligned and held under suction on a 3D automated translation stage system including translation stages during the laser welding process. Next, via the operation 508, a laser spot from a laser source 652 is focused on a first interface between the top plate 602 and the bottom plate 604. Using alignment markers as a reference on the bottom plate 604, the translation stages move in a fixed 2D pattern, with the laser source 652 activated, thereby causing the bottom plate 604 to be welded to the top plate 602 along each periphery of the macro-wells 606, in a square pattern. Next, the laser spot from the laser source 852 is focused on a second interface between the top plate 602 and the bottom plate 604. Using alignment markers as a reference on the bottom plate 604, the translation stages move in another fixed 2D pattern with respect to the laser spot, thereby causing the bottom plate 604 to be further welded to the top plate 602 along each of the reservoir peripheries, in a square pattern. In this manner, the top plate 602 and the bottom plate 604 are fluidly sealed to each other, and the MWAP assembly 600, with the well grid 656, is formed.

### Image Analysis for Visualization of Cells

The present inventive concept utilizes a Fluorescence Scanning Electron Microscope (SEM) to take micrographs to examine the cells in each of the micro-wells 130, 630. Images are taken, via the SEM, when the micro-well array 128, 628 includes one or more fluorescently-labeled tumor cells. For example, FIG. 1E is exemplary of a fluorescence image of the micro-well array 128 with sixteen (16) micro-wells 130. The tumor cells 134A, 134B, are fluorescently labeled tumor cells, which have been placed and are being stored within the micro-well array 128. SEM or other analytical tools, enable the user to visualize cell growths and/or divisions of the cells under various kinds of drugs. In this manner, the micro-wells 130, 630 advantageously enable the user of the MWAP assembly 100, 600 to study how the one or more tumor cells 134A, 1348, 134C respond to the varying degrees of the 3D spatial confinement.

### Applications

Drug development is a time-consuming and prohibitively-expensive process. High failure rates of tumor drugs can be attributed, in part, to poor selectivity of drug molecules during in-vitro screening. The MWAP assembly 100, 600 significantly improves in-vitro drug screening sensitivity of tumor drugs.

The MWAP assembly 100, 600 facilitates drug screening applications where high throughput and high content capability are beneficial, for example, when a large library of drug molecules need to be screened. The MWAP assembly 100 with 3000 to 4000 of the micro-wells 130 is particularly designed to provide an optically clear bottom plate or window, enable high throughput, and be compatible with high content plate imagers for drug screening assays.

The MWAP assembly 100, 600 is operable to spatially confine single cells or multiple cells in the micro-wells 130, 630 that have varying degrees of spatial confinement in size and symmetry. The degree of spatial confinement in size and symmetry may vary with shapes and sizes of the micro-wells 130, 630. The micro-wells 130, 630 can create a spatial confinement, which may have a plurality of functions that can vary for a particular application.

When one or more of the micro-wells 130, 630 is small enough, a single tumor cell or a multicellular cell can be spatially confined in the micro-well 130, 630, for example, by touching the side walls and the bottom wall of the micro-well 130, 630, the physical confinement of the micro-well 130, 630 can provide 3D confinement for the cells.

In addition to the spatial confinement, the symmetry of confinement space of the micro-wells 130, 630 has a profound effect on the ability of a cell to divide. The micro-wells 130, 630 trap single ones of the cells and introduce varying degrees of confinement symmetry, which in turn yield varying results. The confinement symmetry of the micro-wells 130, 630 may range from symmetrical (dorsal-ventral and anterior-posterior) square wells (cuboid) with straight walls to asymmetrical triangular well (e.g. pyramid) with slanted walls.

It is foreseen that tumor cells may need a symmetrical space to divide and grow. In some embodiments, one or more of the micro-wells 130, 630 are asymmetric, thereby preventing the tumor cells from dividing and growing. For example, the micro-wells 130, 630 may have a triangular shape or pyramid shape, with varying lengths or equal lengths of each side.

In some embodiments, cell culture protocols are provided.

The MWAP assembly 100, 600 is designed for, but not limited to, three cancer related applications, that is, 1) Dormancy Assay (single cell confinement in varying degrees of symmetries), 2) Spheroid Assay (standardized size of clonal spheroids), and 3) Clonogenic Assay.

### Dormancy Assay

Dormant cells are responsible for recurrence of cancer years after treatment at the primary site and/or metastasized secondary site. These dormant cells escape initial treatment as dormant cells are in some form of cell cycle arrest, e.g., idling mode, and hence are resistant to chemotherapy or radiation therapy that targets dividing cells given the dormant cells are not dividing. One of the challenges to formulate a drug that targets dormant cells is lack of in-vitro or in-vivo models to test the drug. No conventional technology is able to spatially induce dormancy of tumor cells and test drugs.

The MWAP assembly 100, 600 is operable to spatially induce dormancy of tumor cells and to test drugs, including anti-dormancy drugs, as a Dormancy Assay, which includes the micro-well array 128, 628 of having various shapes and spatial confinement, and can induce dormancy of single tumor cells. It is foreseen that use of symmetry on cell division as an assay, via the present inventive concept, may also induce dormancy of varying degrees in tumor cells.

Numerous anti-dormancy drugs can be introduced into one or more of the macro-wells 106, 606 of the top plate 102, 602 along with the single tumor cells. Then, different ones of the micro-wells 130, 630 of various shapes and spatial confinement of the bottom plate 104, 604 can be used to test the numerous anti-dormancy drugs. In this manner, the MWAP assembly 100, 600 can be used to determine which drug is more effective in inducing dormancy of the single tumor cell.

It is foreseen that single cell confinement in smaller ones of the micro-wells 130, 630 can serve functions including isolation and induction of dormancy depending on degree of spatial confinement. It is foreseen that small dimensional ones of the micro-wells 130, 630, e.g., having 8 μm to 25 μm for the cross-section dimensions, may be used for single cell confinement or single cell applications.

Experiments were performed on a single cell Dormancy Assay and demonstrated that single tumor cells do not divide in pyramid micro-wells for a single cell Dormancy Assay.

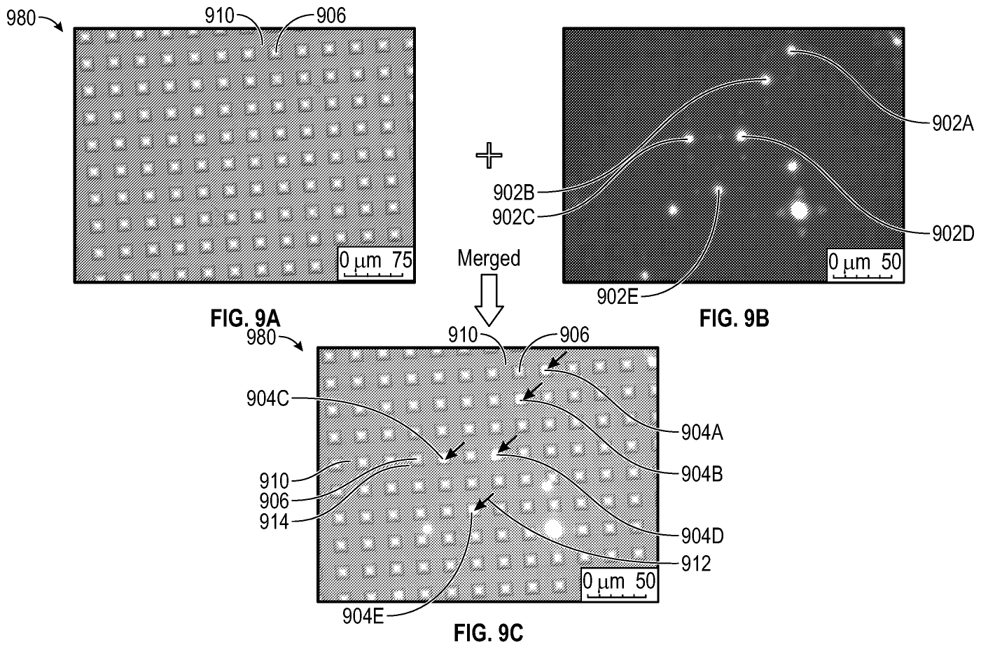

FIG. 9A is a top view of a bottom plate 908 including pyramid micro-wells 906 for a single cell Dormancy Assay, in accordance with embodiments of the present inventive concept. As shown in FIG. 9A, the bottom plate 908 includes a number of the pyramid micro-wells 906 separated or isolated from each other by a dividing wall 910, which extends in a direction perpendicular to the bottom plate 908. The micro-wells 906 include sides 914 having a width of about 10 μm.

FIG. 9B is a fluorescence image showing single tumor cells in the bottom plate 908 for the single cell Dormancy Assay of FIG. 9A, in accordance with embodiments of the present inventive concept. As shown in FIG. 9B, bright spots 902A-E represent single tumor cells.

FIG. 9C is a top view of the bottom plate 908 including the single tumor cells 902A-E trapped in the pyramid micro-wells 908, in accordance with embodiments of the present inventive concept. FIG. 9C is a view of FIG. 9A and FIG. 9B merged together. As shown in FIG. 9C, single tumor cells 902A-E are trapped in the pyramid micro-wells 906, as pointed to by arrows 912. The single tumor cells 902A-E did not divide after six days post plating, i.e., introducing the single tumor cells 902A-E into the pyramid micro-wells 908. Notable, a typical doubling time is thirty-six hours. It will be appreciated by those skilled the art that the shape and/or size of the pyramid micro-wells 908 may vary for the single cell Dormancy Assays without deviating from the scope of the present inventive concept.

### Clonogenic Assay

A Clonogenic Assay is commonly used to quantify effect of tumor drugs and radiation therapy to disrupt growth of a single tumor cell into a colony of clonal cells, e.g., 50 clonal cells or more. Conventional well plates are traditionally used for the assay by seeding each well with a single cell. There are several drawbacks of using conventional well plates including that the conventional wells are limited and do not enable a user to test and observe variations. For instance, the conventional wells have a limited number of wells per plate. Also, overlapping colonies become problematic for counting.

The MWAP assembly 100, 600 is designed for use as a Clonogenic Assay. In the Clonogenic Assay, a single tumor cell can be seeded in a square micro-well large enough to hold a number of clonal cells, e.g., 50 or more clonal cells. The tumor cell is quantified based on its ability to form colonies/spheroid before and after drug treatment under adherent and non-adherent surface properties of the micro-well. Drugs and radiation therapy may be able to disrupt growth of the single tumor cell into the colony of clonal cells. Numerous drugs can be introduced into the micro-wells 130, 630, along with the single tumor cell. Then, the micro-wells 130, 630 including the square micro-well 502, the square pyramid micro-well 302, and the pyramid micro-well 402 can be used to test these numerous drugs to see which drug is more effective in disrupting growth of the single tumor cell into the colony of clonal cells.

Experiments were performed on a single cell Clonogenic Assay and demonstrated growth of single clonal cells into colonies of the clonal cells in the single cell Clonogenic Assay.

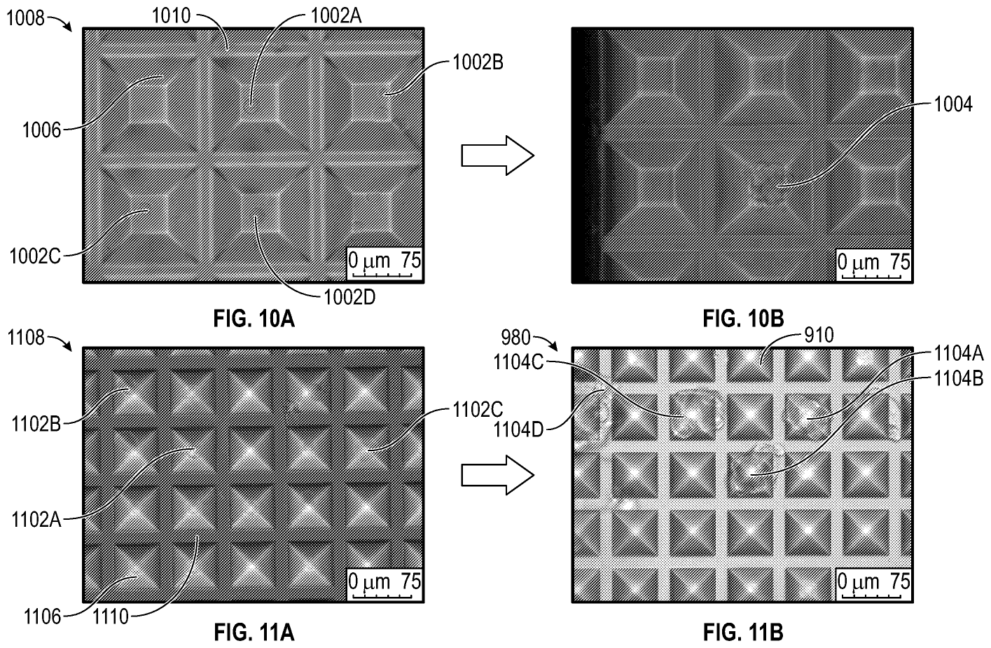

FIG. 10A is a top view of a bottom plate 1008 including square pyramid micro-wells 1006 trapped with single clonal cells for a single cell Clonogenic Assay, in accordance with embodiments of the present inventive concept. As shown in FIG. 10A, the bottom plate 1008 includes a number of the square pyramid micro-wells 1006 separated or isolated from each other by a dividing wall 1010, which extends in a direction perpendicular to the bottom plate 1008. The square pyramid micro-wells 1006 are previously discussed as the square pyramid micro-well 302 and illustrated via FIGS. 3A-3C. As shown in FIG. 10A, single clonal cells 1002A-D were trapped in the square pyramid micro-wells 1006. The single clonal cells 1002A-D are small compared to the size of the micro-wells 1006, such that there is space for the single clonal cells 1002A-D to grow in the micro-wells 1006.

FIG. 10B is a top view of the bottom plate 1008 including the square pyramid micro-wells 1006 trapped with colonies of the clonal cells 1002A-D in the single cell Clonogenic Assay, in accordance with embodiments of the present inventive concept. As shown in FIG. 10B, one clonal cell 1002D was grown into colonies 1004 of the clonal cells 1002A-D, thereby expanding and filling the space provided by the micro-well 1006.

FIG. 11A is a top view of a bottom plate 1108 including pyramid micro-wells 1106 trapped with single clonal cells for a single cell Clonogenic Assay, in accordance with embodiments of the present inventive concept. As shown in FIG. 11A, the bottom plate 1108 includes a number of the pyramid micro-wells 1106 separated or isolated from each other by a dividing wall 1110, which extends in a direction perpendicular to the bottom plate 1108. The pyramid micro-wells 1106 are previously discussed as the pyramid micro-well 402 and illustrated via FIGS. 4A-4C. As shown in FIG. 11A, single clonal cells 1102A-D were trapped in the pyramid micro-wells 1106. The single clonal cells 1102A-D are small compared to the size of the micro-wells 1106, such that there is space for the single clonal cells 1102A-D to grow in the micro-wells 1106.

FIG. 11B is a top view of the bottom plate 1108 including the pyramid micro-wells 1106 trapped with colonies of the clonal cells 1102A-D in the single cell Clonogenic Assay, in accordance with embodiments of the present inventive concept. As shown in FIG. 11B, several clonal cells 1102A-D were grown into colonies 1104A-D of the clonal cells 1102A-D, thereby expanding and filling the space in the micro-wells 1106. It will be appreciated by those skilled the art that the shape and/or size of the pyramid micro-wells 1106 may vary for the single cell Clonogenic Assays without deviating from the scope of the present inventive concept.

### Spheroid Assay

Multi-Cellular Tumor Spheroids (MCTS) are 3D in-vitro models of solid tumor for basic research and drug screening applications. Although there are some conventional platforms available for users to attempt to create MCTS, such conventional platforms are limited and unable to generate clonal MCTS of a fixed size. Indeed, none of the conventional platforms are operable to produce clonal MCTS of a fixed size, or a standardized.

The MWAP assembly 100, 600 is designed as a Spheroid Assay, and is operable to produce a standardized size for MCTS, which can be used for research or drug screen applications. It is foreseen that the multi-cellular confinement in the micro-wells 130, 630 are operable to function to coax and restrict cells into forming spheroids of a fixed size or a standard size. It is foreseen that larger size ones of the micro-wells 130, 630, having 26 μm to 300 μm for the cross-section dimensions, may be used for multi-cellular confinement or applications.

Experiments were performed on a Spheroid Assay and demonstrated growth of a small clonal of breast cancer cells in square pyramid micro-wells of the Spheroid Assay to fill the space in the square pyramid micro-wells to obtain breast cancer cells of a fixed size or a standardized size.

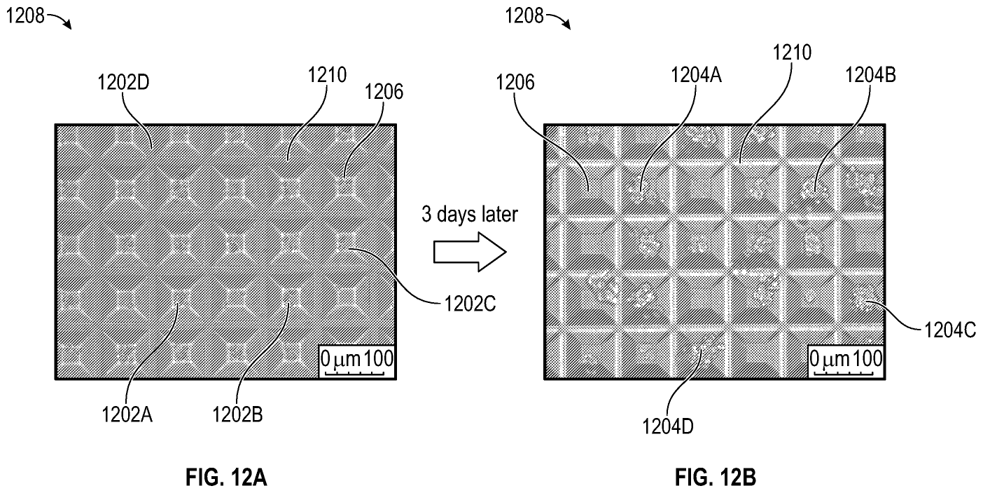

FIG. 12A is a top view of a bottom plate 1208 including square pyramid micro-wells 1206 trapped with breast cancer cells for a Spheroid Assay, in accordance with embodiments of the present inventive concept. As shown in FIG. 12A, the bottom plate 1208 includes a number of the square pyramid micro-wells 1206 separated or isolated from each other by a dividing wall 1210, which extends in a direction perpendicular to the bottom plate 1208. The square pyramid micro-wells 1206 are previously discussed as the square pyramid micro-well 302 and illustrated via FIGS. 3A-3C. As shown in FIG. 12A, multiple breast cancer cells or a small clonal of breast cancer cells 1202A-D were trapped in the square pyramid micro-wells 1206. The small clonal cells 2002A-D were small compared to the size of the square pyramid micro-wells 1206, such that there was space for the small clonal of breast cancer cells 1202A-D to grow in the micro-wells 1206. Also, all the square pyramid micro-wells 1206 had the same size or a fixed size for the Spheroid Assay.

FIG. 12B is a top view of the bottom plate including square pyramid micro-wells trapped with grown breast cancer cells in the Spheroid Assay of FIG. 12A, in accordance with embodiments of the present inventive concept. As shown in FIG. 12B, small clonal of breast cancer cells 1202A-D grow into larger clonal of breast cancer cells 1204A-D after 3 days. With more time, the larger clonal of breast cancer cells could continue to grow until the clonal of breast cancer cells fill the space in the square pyramid micro-cell 1206 to form a clonal of MCTS of a fixed size, which is defined by the square pyramid micro-wells 1206. It will be appreciated by those skilled the art that the shape and/or size of the square pyramid micro-wells 1206 may vary for the Spheroid Assays without deviating from the scope of the present inventive concept.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall therebetween.

What is claimed is:

1. A micro-well array plate (MWAP) assembly comprising:
a top plate having a plurality of macro-wells arranged in an array;
a bottom plate operable to be secured to the top plate, the bottom plate having a plurality of micro-wells; and
a well grid formed when the bottom plate is secured to the top plate, the well grid defined via the plurality of macro-wells and the plurality of micro-wells with each of the plurality of macro-wells isolating a set of the plurality of micro-wells from another set of the plurality of micro-wells.

2. The assembly of claim 1, wherein the plurality of micro-wells are operable to provide varying degrees of three-dimensional (3D) spatial confinement.

3. The assembly of claim 1,
wherein,
each of the plurality of macro-wells includes a cavity surrounded by a perimeter wall extending from a bottom surface of the top plate to a top surface of the top plate, and
two neighboring ones of the plurality of macro-wells share a portion of the perimeter wall.

4. The assembly of claim 3,
wherein,
at least a portion of the plurality of micro-wells includes one or more triangular micro-wells, square pyramids with a bottom surface, or pyramids with a bottom common point to provide asymmetric confinement, and
each of the plurality of micro-wells includes a micro-well perimeter wall with an upper perimeter of a first length equal to or longer than a second length of a lower perimeter.

5. The assembly of claim 1, wherein the isolating causes the set of the plurality of micro-wells to be fluidly sealed from the another set of the plurality of micro-wells.

6. The assembly of claim 1, wherein the well grid is formed when a plurality of lower circumferential perimeters of the macro-wells of the top plate abut to a plurality of upper circumferential perimeters of the plurality of micro-wells of the bottom plate.

7. The assembly of claim 6, wherein each of the plurality of lower circumferential perimeters surrounds one of the plurality of macro-wells.

8. The assembly of claim 6, wherein each of the plurality of upper circumferential perimeters surrounds a different set of the plurality of micro-wells.

9. The assembly of claim 1, wherein the top plate and the bottom plate are permanently bonded together.

10. The assembly of claim 1, wherein at least a portion of the plurality of micro-wells includes straight sidewalls or slanted sidewalls and a same depth.

11. The assembly of claim 1, wherein at least a portion of the plurality of micro-wells has straight sidewalls with bottom surfaces at varying depths.

12. The assembly of claim 1,
wherein,
each of the plurality of micro-wells includes straight sidewalls extending perpendicular to a base with a bottom surface or slanted sidewalls extending oblique to the base without the bottom surface.

13. The assembly of claim 1,
wherein,
each of the plurality of micro-wells is defined by a plurality of sidewalls extending perpendicular to a bottom surface, and
the plurality of sidewalls have varying depths among different ones of the plurality of micro-wells thereby defining varying depths among the different ones of the plurality of micro-wells.

14. The assembly of claim 1, wherein the MWAP assembly is a single cell MWAP assembly operable to spatially induce dormancy of tumor cells as a dormancy assay.

15. The assembly of claim 14, wherein the micro-wells have a cross-section dimension ranging from 8 to 25 μm.

16. The assembly of claim 1, wherein the MWAP assembly is operable to generate clonal multi-cellular tumor spheroids (MCTS) of a fixed size as a spheroid assay.

17. The assembly of claim 16, wherein the micro-wells have a cross-section dimension ranging from 26 to 300 μm.

18. The assembly of claim 1,
wherein,
the MWAP assembly is operable to quantify effects of tumor drugs and radiation therapy to disrupt growth of a single tumor cell with at least one of a plurality of drugs into a colony of clonal cells as a Clonogenic Assay,
each of the plurality of macro-wells is operable to be seeded with the single tumor cell with the at least one of the plurality of drugs, and
the plurality of micro-wells includes 96 sets of micro-wells.

19. The assembly of claim 18, wherein each of the plurality of micro-wells is a square micro-well.

20. The assembly of claim 18, wherein each of the plurality of micro-wells is configured to hold 50 or more clonal cells.

21. The assembly of claim 1, wherein the plurality of micro-wells includes 2000 to 5000 micro-wells such that the MWAP assembly is operable to have a high throughput.

22. A method of fabricating a micro-well array plate assembly, the method comprising:
forming a top plate having a plurality of macro-wells arranged in an array within a frame;
forming a bottom plate having a plurality of arrays of micro-wells operable to provide varying degrees of three-dimensional (3D) spatial confinement; and
forming a well grid by securing the bottom plate to the top plate, the well grid defined via the plurality of macro-wells and the plurality of micro-wells with each of the plurality of macro-wells isolating a set of the plurality of micro-wells from another set of the plurality of micro-wells.

23. The method of claim 22, further comprising:
aligning the top plate and the bottom plate.

24. The method of claim 22, wherein the top plate is formed via an injection molding process.

25. The method of claim 22, wherein the bottom plate is formed via an embossing process.

26. The method of claim 22, wherein the bottom plate is formed using an optically clear or transparent biocompatible cyclic olefin polymer (COP) to enable inspection of an interior of at least the plurality of micro-wells.

27. The method of claim 22, wherein the top plate is formed using a black cyclic olefin polymer (COP).

28. The method of claim 22, wherein the top plate and the bottom plate are secured together to form the micro-well plate via a laser welding process.
